(19) United States
(12) Patent Application Publication
(10) Pub. No.: US 2002/0031556 A1
(43) Pub. Date: Mar. 14, 2002

(54) Stabilized hydrogen peroxide composition and method of making a stabilized hydrogen peroxide composition

(76) Inventor: Ake R. Lindahl, Skurup (SE)

Correspondence Address: MANELLI DENISON & SELTER, 2000 M STREET NW SUITE 700, WASHINGTON, DC, 20036-3307, US

(21) Appl. No.: 09/891,256

(22) Filed: Jun. 27, 2001

Related U.S. Application Data
(60) Provisional application No. 60/219,608, filed on Jul. 21, 2000.

(51) Int. Cl. A61K 33/40

(52) U.S. Cl. 424/616

(57) ABSTRACT

A topical, pharmaceutical composition containing hydrogen peroxide at a concentration of 2 wt. % or less, being stabilized by a combination of saturated polyvalent carboxyacids with a carbon chain length of 2 to 6, salts of tin, pyrophosphate, salicylic acid and monoglycerides of saturated fatty acid in crystalline form and having a pH of 3.5 to 4.9.

This application claims priority to U.S. Ser. No. 60/219,608, filed on Jul. 21, 2000, the complete disclosure of which is incorporated herein by reference.

### FIELD OF THE INVENTION

The present invention relates to a composition and a method for controlling stability and effect of hydrogen peroxide in a topical formulation suitable for delivery of hydrogen peroxide to the skin for the purpose of managing skin diseases and other skin problems. Stability and anti-microbial effects of the hydrogen peroxide is enhanced.

### BACKGROUND OF THE INVENTION

Stabilization of hydrogen peroxide and the importance of the concentration of hydrogen peroxide have been thoroughly described by Shumb, et. al. A general conclusion made by Shumb was that hydrogen peroxide is more stable at high concentrations than at low concentrations. Shumb has also demonstrated that the degradation rate of hydrogen peroxide is dependent on which contaminants are present and what stabilization principles are used. The conditions under which maximum and minimum degradation rates occur are also dependent upon the composition of the formulation. If, for example, iron or chromium are present as contaminants, the variation of degradation rate with pH will differ for each contaminant. The conclusions drawn from results generated with iron as a contaminant generally do not apply when the contaminant is chromium. Thus, when evaluating the stability of hydrogen peroxide, the contaminants in the product and the stabilizers utilized have to be considered. Each combination of contaminants should be regarded as unique and will consequently have their own requirement on stabilizers. Furthermore, requirements on optimal pH will differ accordingly.

The rules and regulations for pharmaceutical products requires that the product should be capable of being stored at +25° C. for a reasonably long distribution and storage period, under which period it should maintain at least 90% of the labeled amount of hydrogen peroxide. The period is usually about 2 years.

Oxalic acid in combination with tin salts for use as a stabilizer is disclosed in U.S. Pat. No. 5,078,672 (Dougherty). The stabilizing effect of oxalic acid and other C2 to C6 saturated polycarboxy acids, in combination with stannates on medium to high concentrated hydrogen peroxide solutions, 6-70%, is described. The preferred mode of application is in the form of a salt, a tin salt of oxalic acid. The role of oxalic acid in this formulation is to stabilize the colloidal fraction of tin, which is a stabilizer for hydrogen peroxide.

Tin in the colloidal state used as a stabilizer for hydrogen peroxide is described by Shumb. The concentrations suggested for stabilization of hydrogen peroxide by Shumb have now been found too be low to be effective in a pharmaceutical formulation. In U.S. Pat. No. 4,534,945 (Hopkins) the same arguments as in Shumb are presented. The amount of tin, proposed in the two publications are based on pure solutions of hydrogen peroxide at high concentrations, namely 20 to 80%. The formulations described in Shumb and the '945 patent contain few contaminants since pure water is utilized. The presence of contaminants will significantly effect the stabilization of hydrogen peroxide.

In other attempts to stabilize hydrogen peroxide, such as GB 1,539,771, GB 2,068,225 and GB 2,076,286 (Fitton), stabilizers have been used. The optimal pH for such combinations is disclosed as 2.5 to 3.2, although a range of 2.5 to 6.5 is disclosed as a broad alternative. The magnitude of stabilization is disclosed in GB '286. The degradation rate of hydrogen peroxide in GB '286, corresponds to 45 to 55% hydrogen peroxide remaining after 2 years storage at +25° C. In U.S. Pat. No. 5,736,582 (Devillez), a pH of 4 to 4.6 is used in the examples, but there is no teaching on the effect of stability of pH. is taught. GB '771 teaches that Hydrogen peroxide can only can be added after manufacture of the carrier composition in order to preserve hydrogen peroxide from degradation during the manufacturing process.

In U.S. Pat. No. 3,954,974 (Hertzog), the optimal pH for an aqueous emulsion was taught as 2.7 to 4.7. It is taught that at pH 5 and higher the stability of hydrogen peroxide is inferior to the preferred interval of 2.7 to 4.7.

Further attempts to stabilize hydrogen peroxide for topical use are described in U.S. Pat. No. 4,812,173 (Tsao) where the optimal pH for hydrogen peroxide stabilization is taught as being 5.5 to 8.0. The stabilizers used in this formulation were phosphonic acids.

There is a need for a topical pharmaceutical-grade composition of hydrogen peroxide which is suitable for application to skin.

### SUMMARY OF THE INVENTION

An objective of the invention is to provide a method of making a pharmaceutical-grade composition of stabilized hydrogen peroxide which is suitable for application to skin.

Another objective of the invention is to provide a pharmaceutical-grade composition of stabilized hydrogen peroxide which is suitable for application to skin.

The above objectives and other objectives are achieved by the methods and compositions disclosed herein. Provided is a method of making a stabilized hydrogen peroxide composition comprising about 2% wt. % or less of hydrogen peroxide based on the total weight of the composition which is suitable for application to human skin. The method comprises:

adding to water a polycarboxylic acid having a chain length of 2 to 6 carbon atoms, a tin salt, salicylic acid or a salt of salicylic acid, and at least one monoglyceride of a fatty acid in crystalline form to form a mixture;

heating the solution to a temperature sufficient to melt said crystalline monoglyceride;

cooling the mixture at a controlled rate to form crystals; and

adjusting the pH to about 3.5 to about 4.9.

The invention also provides a pharmaceutical, hydrogen peroxide composition which is suitable for application to human skin comprising:

about 2 wt. % or less of hydrogen peroxide;

a polycarboxylic acid having a chain length of 2 to 6 carbon atoms;

a tin salt;

salicylic acid or a salt of salicylic acid;

at least one monoglyceride of a fatty acid in crystalline form to form a mixture; and

balance water, wherein the composition has a pH of about 3.5 to about 4.9, and wherein all wt. % are based on the total weight of the composition.

The compositions are capable of maintaining an hydrogen peroxide efficacy of at least 90% after two years of storage at ambient temperatures.

### DETAILED DESCRIPTION OF THE INVENTION

The composition can be formulated by combining the following components with water: a polycarboxylic acid having a chain length of 2 to 6 carbon atoms; a tin salt; salicylic acid or a salt of salicylic acid; and a monoglyceride of a fatty acid in crystalline form, to form a mixture. The solution is then heated to a temperature sufficient to dissolve the crystalline monoglycerides. The solution is cooled at a controlled rate to form crystals of the monoglyceride of a fatty acid and then the pH is adjusted to about 3.5 to about 4.9. It should be noted that unless otherwise mentioned pH values refers to determinations performed immediately after manufacture. Hydrogen peroxide can be added before or after cooling the mixture, as desired for the particular application.

For most applications, it has been found that the solution only need be heated to about 70° C. to melt the crystalline monoglycerides. The cooling can be conducted as desired to provide the desired size crystals. Preferably, the cooling is conducted at a fixed rate of about 0.5 to 5° C. per minute, until crystallisation begins, which is usually about 30 to 39° C. When crystallisation is completed to a desired level, the dispersion can be further cooled.

In formulating the composition, it has surprisingly found that pH in this type of stabiliser composition is of significant importance to the stability of hydrogen peroxide. Thus, the pH of the composition should be adjusted to a level which stabilizes of hydrogen peroxide. In the present stabiliser combination, the optimal pH during storage has been found to be from about 3.5 to about 4.9, preferably about 4.5 to about 4.9. This is in contrast to earlier findings of Shumb and other authors referred to in the background section. It has been found that pH in the composition usually increases after manufacture. A composition that immediately at manufacture has a pH of 4.6 will increase to pH 5 after a few weeks storage and usually remain below pH 5.3 for the remaining shelf life period.

The adjustment of pH in pharmaceutical compositions is now well known and one skilled in the art will easily be able to adjust the pH to the ranges described herein using conventional acids and bases. For example, if the pH is too high, conventional acids can be added. If the pH is too low, conventional bases such as sodium hydroxide can be added.

The composition exhibits significantly enhanced stabilization of the hydrogen peroxide compared to common solutions of hydrogen peroxide. In an experiment performed and disclosed herein, the mean inhibition concentrations hydrogen peroxide was more than 20 times more effective in the present composition at 1 wt. % concentration than in an NF solution diluted with water to 1 wt. %. Furthermore, the composition is capable of maintaining a concentration of hydrogen peroxide efficacy of at least 90% after two years of storage at ambient temperatures. Thus, the composition is capable of meeting or exceeding requirements for use as a topical pharmaceutical agent.

The polycarboxylic acid having a chain length of 2 to 6 carbon atoms is preferably oxalic acid. The acids can be added to the formulation as salts or the acid, as desired. The most preferred acid of this type is oxalic acid. The acids are preferable added in concentrations of from about 0.05 to about 0.5 wt. % and more preferably about 0.1 to about 0.3 wt. %. All weight % are based on the total weight of the composition unless otherwise stated.

Tin, in the form of a salt, for example a sodium salt or a pyrophosphate, can be added in an amount of from about 0.005 to about 0.05 wt. %, more preferably about 0.01 to about 0.03 wt. % corresponding to the amount of tin.

The experimental evidence demonstrates that oxalic acid in the composition acts as a stabiliser for hydrogen peroxide without being combined with tin and also that the combination with tin improves stability.

Salicylic acid can be added in the amount of from about 0.02 to about 0.5 wt. %, preferably about 0.05 to about 0.2 wt. %. Salicylic acid may also be added as a salt of salicylic acid, if desired. Concentrations of salicylic acid above 0.5 wt. % should be avoided since above this level pharmacological effects of salicylic acid could be expected, unless pharmacological effects are desired.

The crystalline monoglyceride can be added in the amount from about 1 to about 35 wt. %. The crystalline monoglyceride preferably has a carbon chain length of from about 10 to about 14. Preferred examples of the crystalline monoglyceride include 1-Glycerolmonolaurate, C12, or 1-Glycerolmonomyristate, C14, or mixtures thereof. The amount of and the ratio between C12 and C14 can be varied depending on the desired viscosity of the final product. For example, the ratio C12:C14 may vary from about 1:3 to 1:1 for a cream product and 1:3 to 1:0 for a lotion/spray form product with lower viscosity. The amount of crystalline monoglyceride in a cream usually varies from about 15 to about 35 wt. % while lotions and sprays preferable have a crystalline monoglyceride content of from about 1 to about 15 wt. %.

Suitable crystalline monoglycerides and the manufacture of formulations containing crystalline monoglycerides are described in U.S. Pat. No. 4,557,935 (Larsson), the complete disclosure of which is incorporated herein by reference. In the '935 patent, the stabilisation of hydrogen peroxide by a solid crystalline lipid dispersion is described. As demonstrated in the example section the stability of formulations manufactured according to the '935 patent are not sufficient in order to fulfil the requirements for use as pharmaceutical products.

Other pharmaceutically acceptable components may be added to the composition. Buffers such as, but not limited to, phosphate buffers and citrate buffers can be introduced. Additional stabilisers such as pyrophosphate and sesquestrants such as but not limited to EDTA and phosphonic acids are also possible to incorporate into the composition. Physical stabilisers, against sedimentation of the crystalline lipids, such as polar surfactants with HLB over and thickeners such as polyacrylic acid derivatives may also be added to the composition to improve its properties. Traditional dermatological agents such as glycerol and propyleneglycol may be added to enhance cosmetic properties.

The invention will be further explained with reference to the following non-limiting examples.

### EXAMPLES

All compositions in the examples were manufactured according to the following method: After heating the water to 70° C. the crystalline lipids were charged and melted. All other components were then added and allowed to dissolve. The mixture was then cooled to 39 to 30° C. at a rate of 0.5 to 5° C. per minute. When the mixture had reached this temperature the cooling was stopped and the product was allowed to crystallise prior to further cooling to 20 to 30° C.

The compositions were manufactured in laboratory scale, except for batch No. 11 which was manufactured in full production scale, 285 kg. Details on the compositions are listed in Tables 1, 2 and 3. The stability of the product was studied by accelerated stability studies at 70° C. 1st order degradation constants were calculated and are listed in Tables 1, 2 and 3. All amounts are in wt. % based on the total weight of the composition.

Compositions According to U.S. Pat. No. 4,557,935, Laboratory Scale Manufacture (up to 10 kg)

TABLE 1

| Components | 1 | 2 |
|---|---|---|
| Monolaurine | 7 | 7 |
| Monomyristine | 21 | 21 |
| H₂O₂ | 1.1 | 1.1 |
| EDTA | — | 0.05 |
| Citric acid | 1 | 1 |
| NaOH | 0.3 | 0.3 |
| Propyleneglycol | — | 2 |
| Myrj 59 | — | 1 |
| Oxalic acid | — | — |
| H₂So₄ to pH | — | — |
| Sodium stannate | — | — |
| Pyrophosphate | — | — |
| Salicylic acid | — | — |
| Water to | 69.6 | 66.55 |
| pH | 3.5 | 3.5 |
| Stability (K1*10⁻⁴) | −23.3 | −3.48 |
| Days to 90% (1) | 60 | 400 |

(1) Days to 90% are calculated by the formula In (Ct/Co)/K1 = t;. The value was compensated for 3% variation in assay and rounded to the nearest 10 days.

Laboratory Scale Manufacture (up to 10 kg)

TABLE 2

| Components | 2 | 3 | 4 |
|---|---|---|---|
| Monolaurine | 7 | 7 | 7 |
| Monomyristine | 21 | 21 | 21 |
| H₂O₂ | 1.1 | 1.1 | 1.1 |
| EDTA | 0.05 | 0.05 | 0.05 |
| Citric acid | 1 | 1 | 1 |
| NaOH | 0.3 | 0.3 | 0.3 |
| Propyleneglycol | 2 | 2 | 2 |
| Myrj 59 | 1 | 1 | 1 |
| Oxalic acid | — | 0.14 | 0.14 |
| H₂So₄ to pH | — | 3.5 | 3.5 |
| Sodium stannate | — | — | 0.025 |
| Pyrophosphate | — | — | 0.025 |
| Water | 66.55 | 62.91 | 62.86 |
| pH during storage | 3.5 | 4 | 4.1 |
| Stability (K1*10⁻⁴) | −3.48 | −2.48 | −2.13 |
| Days to 90% (1) | 400 | 560 | 650 |

Laboratory Scale Manufacture of Batches With Different pH.

TABLE 3

| Components | 5 | 6 | 7 | 8 | 9 | 10 | 11 |
|---|---|---|---|---|---|---|---|
| Hydrogen | 1.15 | 1.15 | 1.15 | 1.15 | 1.15 | 1.15 | 1.15 |
| peroxide | | | | | | | |
| 1-glyceryl-m- | 7 | 7 | 7 | 7 | 7 | 7 | 7 |
| laurate | | | | | | | |
| 1-glyceryl-m- | 21 | 21 | 21 | 21 | 21 | 21 | 21 |
| myristate | | | | | | | |
| Myrj 59 | 1 | 1 | 1 | 1 | 1 | 1 | 1 |
| Propylene glycol | 2 | 2 | 2 | 2 | 2 | 2 | 2 |
| Sodium stannate | 0.04 | 0.04 | 0.04 | 0.04 | 0.04 | 0.04 | 0.04 |
| Salicylic acid | 0.1 | 0.1 | 0.1 | 0.1 | 0.1 | 0.1 | 0.1 |
| Sodium | 0.025 | 0.025 | 0.025 | 0.025 | 0.025 | 0.025 | 0.025 |
| pyrophospate | | | | | | | |
| Sulphuric acid | 0.038 | 0.038 | 0.038 | 0.038 | 0.038 | 0.038 | 0.038 |
| EDTA | 0.05 | 0.05 | 0.05 | 0.05 | 0.05 | 0.05 | 0.05 |
| Oxlaic acid | 0.14 | 0.14 | 0.14 | 0.14 | 0.14 | 0.14 | 0.14 |
| Citric acid | 0.9 | 0.9 | 0.9 | 0.9 | 0.9 | 0.9 | 0.9 |
| Sodium Hydroxide | 0 | 0.194 | 0.032 | 0.392 | 0.465 | .0.557 | 0.032 |
| Purified Water | 66.6 | 66.4 | 66.3 | 66.2 | 66.1 | 66 | 66.3 |
| K1 (*10⁻⁴) | 2.14 | 1.81 | 1.76 | 2.23 | 2.81 | 4.17 | 1.7 |
| Days to 90% (1) | 650 | 770 | 790 | 624 | 500 | 330 | 820 |
| Initial pH | 2.5 | 3.7 | 4.5 | 5 | 5.5 | 6.5 | 4.6 |
| Storage pH | 3.3 | 4.6 | 4.9 | 5.1 | 5.2 | 5.4 | 5.1 |

Composition 1 represents a product described in U.S. Pat. No. 4,557,935 (Larsson) while composition 2 includes an agent known to form complex with iron, EDTA. The degradation rate corresponds to a shelf life of little more than a year for composition 2 while composition 1 degraded much faster (60 days to 90%). The addition of oxalic acid, composition 3, led to a decrease in degradation rate, which was further decreased by the addition of a combination of stannate and pyrophosphate, as in composition 4.

In Table 3 the stability of batches with different pH was demonstrated. Increase of pH up to 4.6 at manufacture and pH 5.1 during storage, led to a further decrease in degradation rate, see Table 3 compositions 5, 6, 7 and 11. Further increase of pH led to faster degradation of hydrogen peroxide as seen in Table 3 compositions. 8, 9, and 10. All these compositions were made in laboratory scale. Compositions 6, 7 and 11 are acceptable for topical pharmaceutical use while compositions 5, 8, 9, and 10 all fail to generate a shelf life of at least 2 years.

It should be noted that the pH during storage differs slightly from that just after manufacture. The storage pH for the most stable compositions 5, 6, 7 and 11 all increased after manufacture and are all inside the limits of about 3.7 to about 5.5. Compositions that are less stable had either a lower storage pH, or a higher storage pH combined with a decrease in pH during storage, see compositions 4 (lower starting and storage pH) and 8, 9, and 10, where pH decreased during storage.

The stabilised compositions 7 and 11, were studied with respect to anti-microbial efficacy. The minimum inhibitory concentration, expressed as No. of dilutions made prior to growth starts, of the composition was compared with that of a solution of hydrogen peroxide and a placebo formulation (without hydrogen peroxide otherwise identical to the present composition) by dilution technique. In Table 4 the results are listed.

No. of Dilutions to MIC (Concentrations Within Brackets)

TABLE 4

| | Cocci | S. Aureus | P. aeruginosa |
|---|---|---|---|
| Present Composition 1% | 256 (0.004) | 128 (0.008) | 512 (0.002) |
| Placebo | 2 | 2 | 2 |
| Hydrogen peroxide | 8 (0.125) | 8 (0.125) | 4 (0.25) |
| solution 1% | | | |

The result demonstrates an increase in effect of the present composition of more than 20 times compared with a solution of hydrogen peroxide. This is in agreement with findings in the '935 patent to Larsson et. al. and demonstrates that the antimicrobial effect of the formulation is maintained although stabilisation of hydrogen peroxide has been performed.

While the claimed invention has been described in detail and with reference to specific embodiments thereof, it will be apparent to one of ordinary skill in the art that various changes and modifications can be made to the claimed invention without departing from the spirit and scope thereof.

1. A method of making a stabilized hydrogen peroxide composition comprising about 2% wt. % or less of hydrogen peroxide based on the total weight of the composition which is suitable for application to human skin, the method comprising:
adding to water about 0.05 to about 0.5 wt. % of polycarboxylic acid having a chain length of 2 to 6 carbon atoms, a tin salt in an amount of about 0.005 to about 0.05 wt. % based on weight of tin, about 0.02 to about 0.5 wt. % of salicylic acid or a salt of salicylic acid, and about 1 to about 35 wt. % of at least one monoglyceride of a fatty acid having a carbon chain length of 12 to 16, in crystalline form to form a solution, wherein all wt. % are based on the total weight of the composition;
heating said solution to a temperature sufficient to melt said crystalline monoglyceride;
cooling said solution at a controlled rate to form crystals; and
adjusting the pH to about 3.5 to about 4.9.

2. A method according to claim 1, wherein said solution is heated to a temperature of about 70° C. to dissolve said crystalline monglyceride.

3. A method according to claim 1, wherein said solution is cooled at a rate of about 0.1 to about 10° C. per minute.

4. A method according to claim 3, wherein said solution is cooled at a fixed rate.

5. A method according to claim 1, wherein said polycarboxylic acid is added in amount of about 0.1 to about 0.3 wt. %; said tin salt is added in an amount of about 0.01 to about 0.03 wt. % by based on the weight of tin; and said salicylic acid is added in an amount of about 0.05 to about 0.2 wt. %.

6. A method according to claim 1, wherein said pH is adjusted to be from about 4.75 to about 4.9.

7. A method according to claim 1, wherein said crystalline monoglyceride comprises 1-Glycerolmonolaurate, C12, and 1-Glycerolmonomyristate, C14.

8. A method according to claim 7, wherein the amount of and the ratio between C12 and C14 are varied depending on desired viscosity of the composition.

9. A method according to claim 7, wherein the ratio C12:C14 is from 1:3 to 1:1 for a cream product and from 1:3 to 1:0 for a lotion or spray form product with lower viscosity.

10. A method according to claim 1, wherein the amount of crystalline monoglycerides is from about 15 to about 35 wt. % when a cream product is desired.

11. A method according to claim 1, wherein the amount of crystalline monoglycerides from about 1 to about 15 wt. % when a lotion or spray product is desired.

12. A method according to claim 1, wherein said polycarboxylic acid comprises oxalic acid.

13. A method according to claim 1, further comprising adding a buffer to said solution.

14. A method according to claim 13, wherein said buffer comprises at least one selected from the group consisting of phosphate buffers and citrate buffers.

15. A method according to claim 1, further comprising adding a stabilizer comprising at least one selected from the group consisting of pyrophosphate and sequestrants.

16. A method according to claim 15, wherein said stabilizer comprises EDTA or phosphonic acid.

17. A method according to claim 1, further comprising adding a physical stabilisers against sedimentation of the lipids.

18. A method according to claim 17, wherein said physical stabilizer comprises a polar surfactant having an HLB over 20.

19. A method according to claim 17, wherein said physical stabilizer comprises a thickener.

20. A method according to claim 19, wherein said thickener comprises a polyacrylic acid derivatives.

21. A method according to claim 1, further comprising adding a dermatological agent.

22. A method according to claim 21, wherein said dermatological agent comprises glycerol or propyleneglycol.

23. A method according to claim 1, wherein said composition retains a hydrogen peroxide efficacy of at least 90% after 2 years.

24. A method according to claim 1, wherein said crystalline monoglyceride has a carbon chain length of from about 10 to about 14.

25. A method of making a stabilized hydrogen peroxide composition comprising about 2% wt. % or less of hydrogen peroxide based on the total weight of the composition which is suitable for application to human skin, the method comprising:
adding to water a polycarboxylic acid having a chain length of 2 to 6 carbon atoms, a tin salt, salicylic acid or a salt of salicylic acid, and at least one monoglyceride of a fatty acid in crystalline form to form a mixture;
heating said solution to a temperature sufficient to melt said crystalline monoglyceride;
cooling said solution at a controlled rate to form crystals; and
adjusting the pH to about 3.5 to about 4.9.

26. A pharmaceutical, hydrogen peroxide composition which is suitable for application to human skin comprising:
about 2 wt. % or less of hydrogen peroxide;
about 0.05 to about 0.5 wt. % of polycarboxylic acid having a chain length of 2 to 6 carbon atoms;
a tin salt in an amount of about 0.005 to about 0.05 wt/% based on weight of tin;
about 0.02 to about 0.5 wt. % of salicylic acid or a salt of salicylic acid;
about 1 to about 35 wt. % of at least one monoglyceride of a fatty acid in crystalline form to form a mixture; and
balance water, wherein said composition has a pH of about 3.5 to about 4.9, and wherein all wt. % are based on the total weight of the composition.

27. A composition according to claim 26, wherein said polycarboxylic acid is present in amount of about 0.1 to about 0.3 wt. %; said tin salt is present in an amount of about 0.01 to about 0.03 wt. % based on the weight of tin; and said salicylic acid is present in an amount of about 0.05 to about 0.2 wt. %.

28. A composition according to claim 26, wherein said pH is from about 4.5 to about 4.9.

29. A composition according to claim 26, wherein said crystalline monoglyceride comprises 1-Glycerolmonolaurate, C12, and 1-Glycerolmonomyristate, C14.

30. A composition according to claim 29, wherein the amount of and the ratio between C12 and C14 depends on desired viscosity of the composition.

31. A composition according to claim 29, wherein the ratio C12:C14 is from 1:3 to 1:1 for a cream product and from 1:3 to 1:0 for a lotion or spray form product with lower viscosity.

32. A composition according to claim 26, wherein the amount of crystalline monoglycerides is from about 15 to about 35 wt. %.

33. A composition according to claim 26, wherein the amount of crystalline monoglycerides from about 1 to about 15 wt. %.

34. A composition according to claim 26, wherein said polycarboxylic acid comprises oxalic acid.

35. A composition according to claim 26, further comprising a buffer.

36. A composition according to claim 35, wherein said buffer comprises at least one selected from the group consisting of phosphate buffers and citrate buffers.

37. A composition according to claim 26, further comprising a stabilizer comprising at least one selected from the group consisting of pyrophosphate and sequestrants

38. A composition according to claim 37, wherein said stabilizer comprises EDTA or phosphonic acid.

39. A composition according to claim 26, further comprising a physical stabilizers against sedimentation of the lipids.

40. A composition according to claim 39, wherein said physical stabilizer comprises a polar surfactant having an HLB over 20.

41. A composition according to claim 39, wherein said physical stabilizer comprises a thickener.

42. A composition according to claim 41, wherein said thickener comprises a polyacrylic acid derivatives.

43. A composition according to claim 26, further comprising a dermatological agent.

44. A composition according to claim 43, wherein said dermatological agent comprises glycerol or propyleneglycol.

45. A composition according to claim 26, wherein said composition retains a hydrogen peroxide efficacy of at least 90% after 2 years.

46. A composition according to claim 26, wherein said crystalline monoglyceride has a carbon chain length of from about 12 to about 16.

47. A pharmaceutical, hydrogen peroxide composition which is suitable for application to human skin comprising:
about 2 wt. % or less of hydrogen peroxide;
a polycarboxylic acid having a chain length of 2 to 6 carbon atoms;
a tin salt;
salicylic acid or a salt of salicylic acid;
at least one monoglyceride of a fatty acid in crystalline form to form a mixture; and
balance water, wherein said composition has a pH of about 3.5 to about 4.9, and wherein all wt. % are based on the total weight of the composition.
